(19) United States
(12) Patent Application Publication
(10) Pub. No.: US 2022/0230764 A1
(43) Pub. Date: Jul. 21, 2022

(54) SYSTEM FOR PATHOGENICITY PREDICTION OF GENOMIC VARIANT USING KNOWLEDGE TRANSFER

(71) Applicant: 3BILLION, Seoul (KR)

(72) Inventors: Kyoungyeul Lee, Seoul (KR); Dhong-gun Won, Seoul (KR)

(73) Assignee: 3BILLION, Seoul (KR)

(21) Appl. No.: 17/644,339

(22) Filed: Dec. 15, 2021

(30) Foreign Application Priority Data
Jan. 19, 2021 (KR) 10-2021-0007219

(51) Int. Cl. G16H 70/60; G06N 3/08; G06N 5/02

(52) U.S. Cl. CPC G16H 70/60; G06N 5/02; G06N 3/08

(57) ABSTRACT

The present disclosure provides a system for Pathogenicity prediction of a genomic variant using knowledge transfer, wherein the system is configured to learn an artificial neural network model using virtual genomic variant data generated from evolutionary conservation data, and learn actual genomic variant data by transferring knowledge by sharing weight values of a hidden layer extracted from the artificial neural network model to the artificial neural network model.

### CROSS-REFERENCE TO RELATED APPLICATION

This application claims a priority under 35 U.S.C. § 119 from Korean Patent Application No. 10-2021-0007219 filed on Jan. 19, 2021, the entire contents of which are hereby incorporated by reference.

### FIELD OF THE INVENTION

The present disclosure relates to a system for Pathogenicity prediction of a genomic variant, and more particularly, to a system for Pathogenicity prediction of a genomic variant using knowledge transfer.

### BACKGROUND OF THE INVENTION

There have been many cases in which genomic variants in the human body cause various diseases and symptoms, but it may be difficult to find the causative gene that causes a disease among numerous genomic variants.

Recently, studies to determine the pathogenicity of genomic variants through automated algorithms to identify disease-causing causative genes have been conducted.

In particular, there have been attempts to determine the pathogenicity of genomic variants through artificial intelligence machine learning. However, in the case of a machine learning model, data dependence is high, and overfitting is highly likely to occur when a machine learning model is trained with only a small amount of clinical and experimental data.

More specifically, the related art predicts pathogenicity through a machine learning model with actual genomic variant data. However, since the amount of clinical and experimental data indicating reliable pathogenicity is limited, overfitting easily occurs when a machine learning model is trained with a small amount of clinical and experimental data.

In particular, it is very difficult to collect data about disease-causing causative genes that cause rare diseases, so it is very important to address these limitations.

### SUMMARY OF THE INVENTION

A technical task of the present disclosure is to provide a system for Pathogenicity prediction of a genomic variant capable of determining pathogenicity of a genomic variant by learning without overfitting through a machine learning model with a small amount of data.

A system for Pathogenicity prediction of a genomic variant using knowledge transfer according to an embodiment of the present disclosure learns an artificial neural network model using virtual genomic variant data generated from evolutionary conservation data, and learns actual genomic variant data by transferring knowledge by sharing weight values of a hidden layer extracted from the artificial neural network model to the artificial neural network model.

The system includes: a virtual genomic variant data generation unit for generating the virtual genomic variant data from the evolutionary conservation data; a virtual variant learning unit for learning an artificial neural network model using the virtual genomic variant data; an actual variant learning unit for learning an artificial neural network model using the actual genomic variant data; and a weight extraction unit for obtaining weight values of a hidden layer of an artificial neural network model when the virtual variant learning unit or the actual variant learning unit learns the artificial neural network model, wherein the actual variant learning unit may apply the extracted weight value to the hidden layer when learning an artificial neural network model.

The virtual genomic variant data generation unit may include: an evolutionary conservation data generation unit for generating the evolutionary conservation data including an evolutionary conservation feature by using multiple sequence alignment (MSA) from target protein sequence data and a plurality of similar pieces of protein sequence data; and a virtual pathogenic variant determination unit for generating each of virtual pathogenic genomic variant data and virtual non-pathogenic genomic variant data according to preset criteria from the evolutionary conservation feature.

The evolutionary conservation feature may be a frequency of amino acids found at a corresponding residue.

The multiple sequence alignment may be performed by a BLAST algorithm or an HHBLits algorithm.

The evolutionary conservation data is an N×21-dimensional feature matrix, where N is an arbitrary number corresponding to a length of an amino acid sequence.

The actual genomic variant data may include actual pathogenic genomic variant data and actual non-pathogenic genomic variant data.

The knowledge transfer may include transfer learning and multi-task learning.

In the transfer learning, after the virtual variant learning unit learns the virtual genomic variant data using an artificial neural network model, weight values extracted by the weight extraction unit may be used by the actual variant learning unit.

In the multi-task learning, the respective weight values extracted from the virtual variant learning unit and the actual variant learning unit may be alternately applied to a hidden layer of an artificial neural network model.

The hidden layer may be an initial layer of the artificial neural network model.

The system may further include a pathogenic determination unit that determines pathogenicity of a target genomic variant using an artificial neural network model learned by the actual variant learning unit.

In addition to the technical tasks of the present disclosure mentioned above, other features and advantages of the present disclosure will be described below, or will be clearly understood by those skilled in the technical field to which the present disclosure pertains from such description and explanation.

According to the present disclosure as described above, there are the following advantages.

According to the present disclosure, it is possible to accurately predict the pathogenicity of a genomic variant that causes a change in a protein sequence by learning without overfitting through a machine learning model with a small amount of genomic variant data.

In addition, the present disclosure uses virtual genomic variant data generated from evolutionary conservation data and transfers knowledge of the weight value of the hidden layer extracted from the artificial neural network model. Hence, only with a small amount of actual genomic variant data, it is possible to perform learning without overfitting by extracting features important for Pathogenicity prediction from genomic variants, thereby accurately Pathogenicity prediction for genomic variants that cause changes in protein sequences.

In addition, other features and advantages of the present disclosure may be newly recognized through embodiments of the present disclosure.

### BRIEF DESCRIPTION OF THE DRAWINGS

FIG. 1 is a block diagram illustrating a schematic configuration of a system for Pathogenicity prediction of a genomic variant using knowledge transfer according to an embodiment of the present disclosure;

FIG. 2 is a block diagram illustrating a schematic configuration diagram of a virtual genomic variant data generation unit according to an embodiment of the present disclosure;

FIG. 3 is a diagram illustrating protein sequence data according to an embodiment of the present disclosure;

FIG. 4 is a diagram illustrating evolutionary conservation data showing an evolutionary conservation feature using multiple sequence alignment according to an embodiment of the present disclosure;

FIG. 5 is a diagram illustrating virtual pathogenic genomic variant data and virtual non-pathogenic genomic variant data according to an embodiment of the present disclosure;

FIG. 6 is a diagram illustrating transfer learning by the system for Pathogenicity prediction of a genomic variant according to an embodiment of the present disclosure;

FIG. 7 is a diagram illustrating multi-task learning in a system for Pathogenicity prediction of a genomic variant according to another embodiment of the present disclosure; and

FIG. 8 is a diagram illustrating that the system for Pathogenicity prediction of a genomic variant according to an embodiment of the present disclosure determines the pathogenicity of a target genomic variant using an artificial neural network model.

### DETAILED DESCRIPTION OF THE INVENTION

In the present specification, in adding reference numerals for elements in each drawing, it should be noted that like reference numerals already used to denote like elements in other drawings are used for elements wherever possible.

The terms described in the present specification should be understood as follows.

It should be understood that the terms “comprise” or “have” do not preclude the presence or addition of one or more other features, integers, steps, operations, components, elements, or combinations thereof.

In addition, hereinafter, the terms to be used herein are defined hereafter to make the specification clear.

As used herein, the term “genomic variant” may refer to a variant in a nucleotide sequence occurring in a chromosome due to various factors. For example, the genomic variant may be a somatic variant, a nucleotide sequence variant due to contamination of a sample, and a nucleotide sequence variant due to a genetic disease. However, the genomic variant is not limited to the above.

Hereinafter, preferred embodiments of the present disclosure will be described in detail with reference to the accompanying drawings.

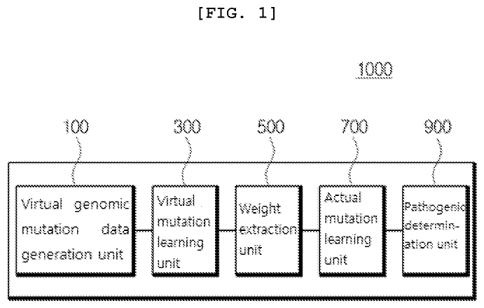

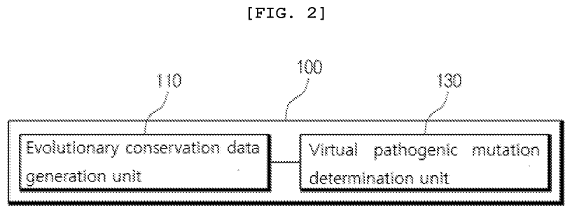

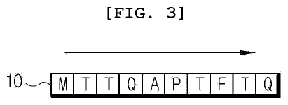

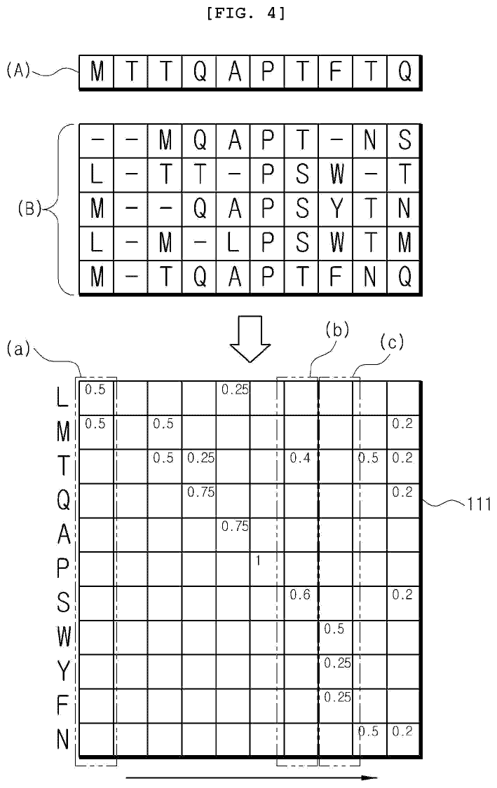

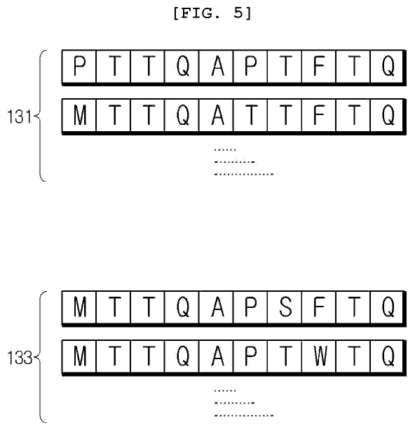

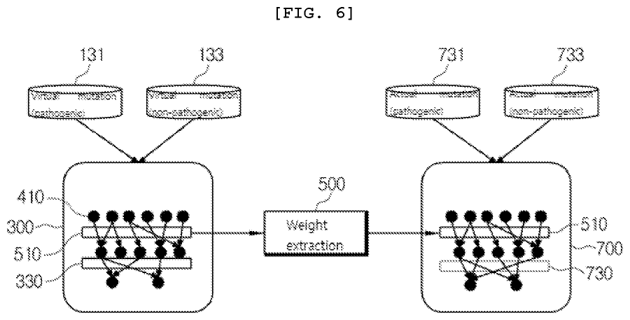

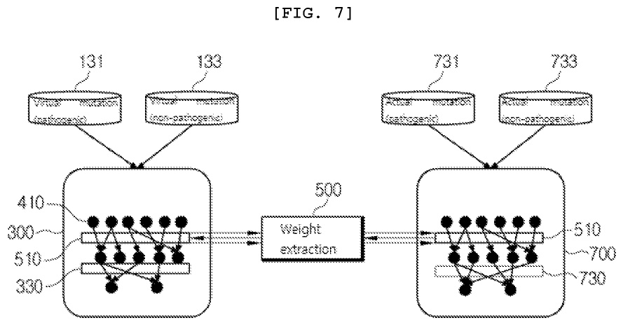

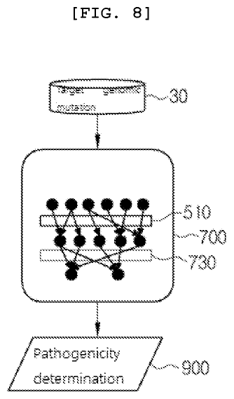

FIG. 1 is a block diagram illustrating a schematic configuration of a system for Pathogenicity prediction of a genomic variant using knowledge transfer according to an embodiment of the present disclosure. FIG. 2 is a block diagram illustrating a schematic configuration diagram of a virtual genomic variant data generation unit according to an embodiment of the present disclosure. FIG. 3 is a diagram illustrating protein sequence data according to an embodiment of the present disclosure. FIG. 4 is a diagram illustrating evolutionary conservation data showing an evolutionary conservation feature using multiple sequence alignment according to an embodiment of the present disclosure. FIG. 5 is a diagram illustrating virtual pathogenic genomic variant data and virtual non-pathogenic genomic variant data according to an embodiment of the present disclosure. FIG. 6 is a diagram illustrating transfer learning by the system for Pathogenicity prediction of a genomic variant according to an embodiment of the present disclosure. FIG. 7 is a diagram illustrating multi-task learning in a system for Pathogenicity prediction of a genomic variant according to another embodiment of the present disclosure. FIG. 8 is a diagram illustrating that the system for Pathogenicity prediction of a genomic variant according to an embodiment of the present disclosure determines the pathogenicity of a target genomic variant using an artificial neural network model.

Referring to FIG. 1, a system 1000 for Pathogenicity prediction of a genomic variant using knowledge transfer according to an embodiment of the present disclosure includes a virtual genomic variant data generation unit 100, a virtual variant learning unit 300, a weight extraction unit 500, an actual variant learning unit 700, and a pathogenic determination unit 900.

The system for Pathogenicity prediction of a genomic variant using knowledge transfer according to an embodiment of the present disclosure may generate virtual genomic variant data used in an artificial neural network model using evolutionary conservation data by a virtual genomic variant data generation unit. While learning the artificial neural network model using the virtual genomic variant data by the virtual variant learning unit, the system may transfer the weights of the hidden layer extracted by the weight extraction unit to the artificial neural network model of the actual variant learning unit to learn actual genomic variant data.

The virtual genomic variant data generation unit 100 may generate evolutionary conservation data and virtual genomic variant data.

Referring to FIG. 2, the virtual genomic variant data generation unit 100 includes an evolutionary conservation data generation unit 110 and a virtual pathogenic variant determination unit 130.

The evolutionary conservation data generation unit 110 may generate evolutionary conservation data including an evolutionary conservation feature by using multiple sequence alignment (MSA) from target protein sequence data and a plurality of similar pieces of protein sequence data.

Referring to FIG. 3, since the total length of the protein sequence is very diverse, only the protein sequence data of an arbitrary specific area may be used according to an embodiment.

Protein sequence data may be expressed as a character string in which a plurality of amino acids (alphabetic words) are sequenced and connected. In this case, arrows indicate the sequence of residues in the protein.

The protein sequence data 10 illustrated in FIG. 3 starts with the amino acid M at the 1st residue and ends with the amino acid Q at the 10th residue.

The evolutionary conservation data generation unit 110 may generate evolutionary conservation data including an evolutionary conservation feature by using multiple sequence alignment (MSA) with the evolutionary conservation data.

Referring to FIG. 4, the evolutionary conservation data generation unit 110 may sequence-align target protein sequence data (A) and a plurality of similar pieces of protein sequence data (B) using multiple sequence alignment (MSA).

First, a target protein may be set, and another protein having a similar sequence to a target protein may be selected as a similar protein. In general, it is assumed that proteins with similar sequences are proteins differentiated in the course of evolution.

A similar protein may be selected by determining the degree of similarity between a target protein and another protein, and the degree of similarity may be determined based on amino acid identity and sequence matching coverage. A number of scoring techniques such as E-value for determining the degree of similarity have been developed.

Since amino acids are changed to other amino acids or new amino acids are added or deleted in the course of evolution, residues at the same position may have different amino acids. When there is no matching residue, a “gap (-)” is indicated.

When the same amino acid is repeatedly found in multiple proteins, the residue is said to be “highly conserved.”

In addition, a multiple sequence alignment of a target protein and a plurality of similar proteins may be performed.

The sequence alignment refers to aligning residues of different proteins so that their positions match, and when there are multiple proteins to be aligned, it is called multiple sequence alignment.

The evolutionary conservation data generation unit 110 according to an embodiment of the present disclosure may perform multiple sequence alignment by a BLAST algorithm or an HHBLits algorithm.

In addition, the evolutionary conservation data generation unit 110 may generate evolutionary conservation data 111 by using a frequency of amino acids found in the corresponding residue as an evolutionarily conservation feature.

In other words, the evolutionary conservation data 111 is sequence data representing evolutionary conservation feature, and may be an N×21-dimensional feature matrix. Here, N is an arbitrary number corresponding to a length of the amino acid sequence. Since there are 21 types of amino acids, it may be expressed in 21 dimensions.

The virtual pathogenic variant determination unit 130 may generate virtual pathogenic genomic variant data and virtual non-pathogenic genomic variant data, respectively, according to preset criteria from the evolutionary conservation feature.

The virtual genomic variant data includes virtual pathogenic genomic variant data and virtual non-pathogenic genomic variant data.

A virtual pathogenic genomic variant is a genomic variant that is rarely found in light of the genetic data of evolutionarily varied species, and a virtual non-pathogenic genomic variant may be a frequently found genomic variant.

Referring to FIG. 4, the proportion of amino acid L in the first residue (a) of the evolutionary conservation data 111 is 50%. When the criterion for determining pathogenicity is preset to 10%, a genomic variant in which amino acid M is changed to amino acid L in the first residue (a) may be considered as non-pathogenic.

Since the proportion of amino acid P in the first residue (a) is 0, a genomic variant in which amino acid M is changed to amino acid P may be considered pathogenic.

Since the amino acid P at the first residue (a) has never been found, it may be thought that when a genomic variant in which the first residue (a) of the target protein is changed to the amino acid P occurs, it will evolutionarily cause a great obstacle to the development of life.

Accordingly, it may be generated as virtual pathogenic genomic variant data that may be considered as high pathogenicity.

Since the proportion of the amino acid S in the 7th residue (b) is 60%, which is 10% or more, the genomic variant in which the amino acid T is changed to the amino acid S may be considered as non-pathogenic.

Since the amino acid S is repeatedly observed at the 7th residue (b), it may be inferred that even when a genomic variant in which the 7th residue of the target protein is changed to the amino acid S occurs, evolutionarily, it does not cause a major obstacle to the development of life.

Accordingly, it can be generated as virtual non-pathogenic genomic variant data that may be considered as low pathogenicity.

Since the proportion of the amino acid W in the 8th residue (c) is 50%, which is 10% or more, the genomic variant in which the amino acid F is changed to the amino acid W may be considered as non-pathogenic.

Referring to FIG. 5, the virtual pathogenic variant determination unit 130 may generate a plurality of virtual pathogenic genomic variant data 131 and virtual non-pathogenic genomic variant data 133, respectively, according to preset criteria from the evolutionary conservation feature.

The virtual variant learning unit 300 may learn the artificial neural network model by using the virtual genomic variant data.

Referring to FIG. 6, the virtual variant learning unit 300 may learn an artificial neural network model using virtual pathogenic genomic variant data 131 and virtual non-pathogenic genomic variant data 133 that are virtual genomic variant data 130.

The system 1000 for Pathogenicity prediction of a genomic variant according to an embodiment of the present disclosure may use a transfer learning technique among knowledge transfer techniques.

In transfer learning, after the virtual variant learning unit 300 completely learns the virtual genomic variant data using the artificial neural network model, the weight extraction unit extracts weight values of a hidden layer in the artificial neural network model of the virtual variant learning unit 300. Then, the artificial neural network model applies the weight value of the hidden layer extracted from the actual variant learning unit to learn the actual genomic variant data.

An Artificial Neural Network model (ANN) may be used, and any one of deep learning networks such as Convolutional Neural Network (CNN), Recurrent Neural Network (RNN), or Transformer may be used.

CNN is one of the most used algorithms in deep learning and can learn sequence data. In this case, it is a method of convolution of nearby residues with one filter.

RNN is the deepest network structure among deep learning by piling up data from every moment in an artificial neural network structure, and is a representative deep learning network that receives sequence data.

In addition, deep learning networks applicable to sequence data such as Transformer, Gated Recurrent Unit (GRU), Long Short-Term Memory (LSTM), Bidirectional Encoder Representations from Transformers (BERT), or XLNET, known as deep learning networks, may be used.

The virtual variant learning unit 300 may generate weights of the sequence feature 410 and the layer 510 while training the artificial neural network model using the virtual genomic variant data 130.

In general, when an artificial neural network model learns data to solve a certain issue, a pattern of data is learned in a plurality of layers 330, 510, and 730.

When the virtual variant learning unit 300 learns the artificial neural network model, the weight extraction unit 500 may obtain weight values 510 of the hidden layer 510 of the artificial neural network model.

The weight extraction unit 500 may extract weight values of the initial layer 510 from among the plurality of layers 330, 510, and 730.

Among the plurality of layers 330, 510, and 730, the weight of the initial layer 510 may be considered to reflect a characteristic important for Pathogenicity prediction in genomic variants, and this may be used for knowledge transfer techniques.

The actual variant learning unit 700 learns the artificial neural network model using the actual genomic variant data, and in this case, the weight value obtained by the weight extraction unit 500 may be used.

In other words, by transferring the weight value of the hidden layer 510 extracted by the weight extraction unit 500 to the artificial neural network model, actual genomic variant data can be learned.

The actual genomic variant data includes actual pathogenic genomic variant data 731 and actual non-pathogenic genomic variant data 733.

The actual variant learning unit 700 may use the weight value of the hidden layer 510 extracted by the weight extraction unit 500 for the initial hidden layer 510 of the artificial neural network model.

With reference to FIG. 7, multi-task learning of a system for Pathogenicity prediction of a genomic variant according to another embodiment of the present disclosure will be described. Except for multi-task learning among knowledge transfer techniques, it is the same as the aforementioned system for Pathogenicity prediction of a genomic variant. Accordingly, the same reference numerals are assigned to the same components, and repeated descriptions of the same components will be omitted.

The system 1000 for Pathogenicity prediction of a genomic variant according to another embodiment of the present disclosure may use multi-task learning among knowledge transfer techniques.

In multi-task learning, the weight values of each hidden layer extracted from the virtual variant learning unit and the actual variant learning unit may be used as a hidden layer of an artificial neural network model by alternating in real time.

For example, it is assumed that the virtual variant learning unit 300 learns by dividing the virtual variant data into A, B, and C, and the actual variant learning unit 700 learns by dividing the actual variant data into D, E, and F.

After the virtual variant learning unit 300 learns the virtual variant data A, the weight extraction unit 500 extracts weight values of the hidden layer 510 of the artificial neural network model. The actual variant learning unit 700 learns the actual variant data D by using a value of the extracted hidden layer weight 510 as a hidden layer 510 weight value of the artificial neural network model.

Next, when the virtual variant learning unit 300 learns the virtual variant data B, the actual variant learning unit 700 learns the actual variant data D, and then learns the virtual variant data B by using the weight value of the hidden layer 510 extracted therefrom.

Again, when the actual variant learning unit 700 learns the actual variant data E, the actual variant learning unit 300 uses the weight value of the hidden layer 510 extracted after learning the virtual variant data B to learn the actual variant data E.

Next, when the virtual variant learning unit 300 learns the virtual variant data C, the actual variant learning unit 700 learns the actual variant data E, and then learns the virtual variant data C by using the weight value of the hidden layer 510 extracted therefrom.

Again, when the actual variant learning unit 700 learns the actual variant data F, the actual variant learning unit 300 uses the weight value of the hidden layer 510 extracted after learning the virtual variant data C to learn the actual variant data F.

As such, the system 1000 for Pathogenicity prediction of a genomic variant according to another embodiment of the present disclosure may use multi-task learning among knowledge transfer techniques.

As a result, the system 1000 for Pathogenicity prediction of a genomic variant according to an embodiment of the present disclosure replaces the data resources required for learning in several layers in the first place with transfer learning or multi-task learning among knowledge transfer techniques. By extracting features important for Pathogenicity prediction of a genomic variant, it is possible to learn without overfitting only with a small amount of actual genomic variant data enough to learn only the late hidden layer 730 of the artificial neural network model.

Referring to FIG. 8, the system 1000 for Pathogenicity prediction of a genomic variant according to an embodiment of the present disclosure further includes a pathogenicity determination unit 900 determining the pathogenicity of a target genomic variant 30 using the artificial neural network model 700 learned in the actual variant learning unit.

The pathogenicity determination unit 900 may display the likelihood of pathogenicity through an activation function using an artificial neural network model as a pathogenicity score between 0 and 1.

The activation function may be a softmax function or a sigmoid function.

As described above, the system 1000 for Pathogenicity prediction of a genomic variant using transfer learning according to an embodiment of the present disclosure generates virtual genomic variant data using protein sequence data and evolutionary conservation data, and transfers the knowledge of the weight values of the hidden layer obtained by learning the virtual genomic variant data as an artificial neural network model, thereby enabling a learning without overfitting only with a small amount of actual genomic variant data.

In other words, the system 1000 for Pathogenicity prediction of a genomic variant using transfer learning according to an embodiment of the present disclosure may perform learning without overfitting through a machine learning model with a small amount of genomic variant data, thereby accurately Pathogenicity prediction for genomic variants that cause changes in protein sequences.

Although the embodiments of the present disclosure have been described with reference to the attached drawings, those skilled in the technical field to which the present disclosure pertains will understand that the present disclosure may be practiced in other detailed forms without departing from the technical spirit or essential features of the present disclosure. Therefore, it should be understood that the above-described embodiments are exemplary in all aspects rather than being restrictive.

The present disclosure described above is not limited to the above-described embodiments and the accompanying drawings. It will be apparent to those skilled in the technical field to which the present disclosure pertains that various substitutions, modifications and changes are possible without departing from the technical spirit of the present disclosure.

1. A system for Pathogenicity prediction of a genomic variant using knowledge transfer, wherein the system is configured to learn an artificial neural network model using virtual genomic variant data generated from evolutionary conservation data, and learn actual genomic variant data by transferring knowledge by sharing weight values of a hidden layer extracted from the artificial neural network model to the artificial neural network model.

2. The system of claim 1, wherein the system comprises:
a virtual genomic variant data generation unit for generating the virtual genomic variant data from the evolutionary conservation data;
a virtual variant learning unit for learning an artificial neural network model using the virtual genomic variant data;
an actual variant learning unit for learning an artificial neural network model using the actual genomic variant data; and
a weight extraction unit for obtaining weight values of a hidden layer of the artificial neural network model when the virtual variant learning unit or the actual variant learning unit learns the artificial neural network model,
wherein the actual variant learning unit applies the extracted weight value to the hidden layer when learning an artificial neural network model.

3. The system of claim 1, wherein the virtual genomic variant data generation unit comprises:
an evolutionary conservation data generation unit for generating the evolutionary conservation data including an evolutionary conservation feature by using multiple sequence alignment (MSA) from target protein sequence data and a plurality of similar pieces of protein sequence data; and
a virtual pathogenic variant determination unit for generating each of virtual pathogenic genomic variant data and virtual non-pathogenic genomic variant data according to preset criteria from the evolutionary conservation feature.

4. The system of claim 3, wherein the evolutionary conservation feature is a frequency of amino acids found in a corresponding residue.

5. The system of claim 3, wherein the multiple sequence alignment is performed by a BLAST algorithm or an HHBLits algorithm.

6. The system of claim 3, wherein the evolutionary conservation data is an N×21-dimensional feature matrix, and the N is an arbitrary number corresponding to a length of an amino acid sequence.

7. The system of claim 2, wherein the actual genomic variant data comprises actual pathogenic genomic variant data and actual non-pathogenic genomic variant data.

8. The system of claim 2, wherein the knowledge transfer comprises transfer learning and multi-task learning.

9. The system of claim 8, wherein in the transfer learning, after the virtual variant learning unit learns the virtual genomic variant data using an artificial neural network model, weight values extracted by the weight extraction unit is used by the actual variant learning unit.

10. The system of claim 8, wherein in the multi-task learning, the respective weight values extracted from the virtual variant learning unit and the actual variant learning unit are alternately applied to a hidden layer of an artificial neural network model.

11. The system of claim 2, wherein the hidden layer is an initial layer of the artificial neural network model.

12. The system of claim 2, wherein further comprising a pathogenic determination unit that determines pathogenicity of a target genomic variant using an artificial neural network model learned by the actual variant learning unit.
